(19) United States
(12) Patent Application Publication
(10) Pub. No.: US 2008/0090297 A1
(43) Pub. Date: Apr. 17, 2008

(54) Methods for determining the metal bioavailability of metal sources

(75) Inventor: James David Richards, Foristell, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Novus International Inc., St. Louis, MO (US)

(21) Appl. No.: 11/580,763

(22) Filed: Oct. 13, 2006

(51) Int. Cl. G01N 33/20

(52) U.S. Cl. 436/80

(57) ABSTRACT

The invention provides methods for determining the relative metal bioavailability of a metal source in an animal or group of animals. The method may be utilized to compare the relative metal bioavailability between different metal sources and it may be used to determine the relative nutritional status of a metal in an animal or group of animals.

### FIELD OF THE INVENTION

The present invention provides methods for determining relative metal bioavailability of a metal source in an animal or a group of animals, for comparing the relative metal bioavailability between different metal sources, and for determining the relative metal nutritional status of an animal.

### BACKGROUND OF THE INVENTION

Animals need nutrients for proper growth and development, to fight infection and disease, promote general health, and achieve other desirable results. Common nutrients, such as essential minerals including calcium, chromium, cobalt, copper, iron, magnesium, manganese, potassium, selenium, and zinc, are often administered in the form of feed supplements to poultry, swine, ruminants, and companion animals.

To be effective, a supplement must deliver the intended mineral in a readily absorbable form. A variety of factors, however, such as mineral solubility, stability in solution, or the presence of competing substances, can affect the bioavailability of many essential minerals. Essential minerals supplied as inorganic salts are frequently poorly absorbed in the gut of many animals, whereas those supplied as chelates or complexes tend to be more readily absorbed. The bioavailability of such complexes, however, is affected by numerous physical and biochemical parameters, such as stability in solution, stability at acidic pH, the point of absorption, competitive absorption by bacteria residing within the gastrointestinal tract, and the formation of insoluble complexes. Irrespective of the form in which the mineral is administered to the animal, if it isn't readily absorbable, the animal may experience a detrimental metal deficiency.

Current methods to measure the bioavailability of a mineral in an animal are plagued by drawbacks. For example, in the agricultural animal industry, the standard assay utilized to determine zinc status is to measure tissue zinc, and more particularly bone zinc levels or liver zinc levels. While this method provides an indication of bioavailability, measuring bone or liver zinc is an imprecise means for accessing zinc bioavailability because most mineral absorption occurs in the gastrointestinal tract of the animal. A need exists for a sensitive assay to rapidly monitor the mineral status of an animal.

### SUMMARY OF THE INVENTION

One aspect of the invention provides a method for determining relative metal bioavailability of a metal source in at least one animal. The method comprises administering the metal source to the animal, detecting the level of expression of a metal responsive biomarker in a sample obtained from the animal and a control sample, and comparing the level of biomarker expression in each sample. In general, a difference in the level of expression between the two samples indicates the relative bioavailability of the metal source in the animal.

A further aspect of the invention encompasses a method for comparing the relative metal bioavailability between a first metal source and a second metal source. The method comprises administering a first metal source to a first animal and a second metal source to a second animal; detecting the level of expression of a metal responsive biomarker present in a first sample obtained from the first animal and in a second sample obtained from the second animal; and comparing the level of biomarker expression in each sample. Typically, a difference in the level of expression of the biomarker indicates that the first and second metal sources have different relative metal bioavailability.

Yet another aspect of the invention provides a method for determining the relative nutritional status of zinc or copper in an animal. The method comprises detecting the level of metallothionein mRNA expression by quantitative real time polymerase chain reaction in a sample obtained from the animal and in a control sample, and comparing the level of metallothionein expression in each sample. Generally speaking, a lower level of metallothionein mRNA in the sample from the animal versus the control sample indicates that the animal may have a zinc or copper deficiency.

Other aspects and features of the invention are provided in more detail herein.

### DESCRIPTION OF THE FIGURES

FIG. 1 illustrates the zinc-dependent increase in metallothionein (MT) mRNA levels in the various chicken tissues. Relative amounts of MT mRNA are plotted as a function of diet and tissue. Each bar represents the expression in one bird.

FIG. 2 illustrates the time course of zinc-dependent induction of metallothionein (MT) mRNA in chicken jejunum. Plotted is the relative amount of MT mRNA as a function of treatment and time. Each bar represents the average MT expression value for three birds. Bars lacking a common superscript are significantly different (P≦0.05).

FIG. 3 illustrates the bioavailability of different zinc compositions in chicken jejunum mucosal scrapings. Relative metallothionein (MT) mRNA levels are plotted as a function of zinc composition. Each bar represents the average MT expression value for six birds. Bars lacking a common superscript are significantly different (P≦0.05).

FIG. 4 illustrates increased levels of liver metallothionein (MT) mRNA in lactating multiparous dairy cows after metal exposure. Plotted is the relative amount of MT mRNA as a function of treatment and time. There were 10 cows per treatment. Bars lacking a common superscript are significantly different (P≦0.05).

### DETAILED DESCRIPTION OF THE INVENTION

The present invention is based upon the discovery that metal bioavailability of a metal source can accurately be determined by monitoring the level of expression of a metal responsive biomarker in an animal. Generally, the level of biomarker expression in response to a metal source can be evaluated using a sensitive detection means, such as quantitative real time polymerase chain reaction, and these data may be used in methods for determining relative metal bioavailability of a metal source in an animal, for comparing the relative metal bioavailability between different metal sources, and for determining the relative metal nutritional status of an animal. Irrespective of the method's particular application, it typically involves administering a metal source to at least one animal; detecting the response of a biomarker to the metal source in a sample obtained from the animal and one obtained from either a control sample or from another animal; and comparing the expression level of the biomarker in the sample taken from the animal versus the sample taken from either the control sample or the other animal. A difference in the level of biomarker expression between the samples generally indicates the relative bioavailability of the metal source in the animal. Each of these parameters utilized in the methods of the invention is described in more detail below.

### (I) Administration of a Metal Source to an Animal

One aspect of the method of the invention comprises administering a metal source to an animal to determine the relative in vivo bioavailability of the metal source. The method may be utilized to determine the relative bioavailability of a metal source in a range of different animals. The animal may be an agricultural animal. Suitable examples include, but are not limited to, chicken, beef cattle, dairy cattle, swine, sheep, goat, horse, duck, turkey, and goose. The animal may be a companion animal, such as cat, rabbit, rat, hamster, parrot, horse, or dog. The animal may also be an aquatic animal, such as fish or shellfish. Non-limiting examples of suitable fish include catfish, tuna, salmon, bass, tilapia, redfish, muskie, pike, bowfin, gar, paddlefish, sturgeon, bream, carp, trout, walleye, snakehead, and crappie. Examples of suitable shellfish include crabs, clams, shrimp, mollusks, octopus, oysters, scallops, and squid. Alternatively, the animal may be a game animal or a wild animal. Non-limiting examples of suitable game animals include buffalo, deer, elk, moose, reindeer, caribou, antelope, rabbit, squirrel, beaver, muskrat, opossum, raccoon, armadillo, porcupine, pheasant quail, and snake. In an exemplary embodiment, the animal is an agricultural animal.

(a) Type of Metal Source

It is envisioned that the relative bioavailability of several types of metal sources may be determined by the practice of the invention to the extent that the metal source, when administered to the animal, causes a detectable modulation in the expression of biomarker. In this context, the term “metal source” is used in its broadest sense to encompass elements, compounds, and compositions that are metallic in nature. In an exemplary embodiment, the metal source is a compound or composition comprising a mineral or metal. Non-limiting examples of minerals and metals include calcium, chromium, cobalt, copper, fluorine, iodine, iron, magnesium, manganese, molybdenum, phosphorus, potassium, selenium, sodium and zinc, which are essential for human and animal health. Particularly important mineral supplements for agricultural animals include, but are not limited to, zinc, copper, iron, manganese, magnesium, calcium, potassium, cobalt, chromium, and selenium.

A metal source, as detailed above, may be provided as a composition or compound comprising a metal in the form of a salt, a complex, or a chelate. In one embodiment, the metal composition may comprise zinc. The zinc composition may be an inorganic zinc salt. Suitable zinc salts include, without limitation, zinc acetate, zinc citrate, zinc chloride, zinc oxide, or zinc sulfate. The zinc composition may be an organic zinc complex. The organic zinc complex may a zinc amino acid complex, such as zinc aspartate, zinc cysteinate, zinc glutamate, zinc glycinate, zinc histidinate, zinc lysinate, zinc methionate, zinc taurinate, or zinc complexed to a mixture of amino acids. The zinc complex may also be a zinc carboxylic acid complex, such as zinc formate, zinc lactate, zinc malate, zinc oxalate, or zinc succinate. The zinc complex may also be a zinc nucleotide complex, such as zinc adenine or zinc inosine. Other suitable zinc complexes include zinc polysaccharide complexes and zinc proteinate complexes. The zinc composition may also be a zinc chelate. Suitable chelates include zinc bicine, zinc CDTA, zinc 1,2-diaminoethane, zinc dimercaprol, zinc dithizone, zinc DTPA, zinc EDTA, zinc EGTA, zinc oxine, and zinc 1,10-phenanthroline.

In another embodiment, the metal source may comprise copper. The copper composition may be a copper salt, such as copper acetate, copper carbonate, copper chloride, copper iodide, copper nitrate, copper oxide, copper selenite, copper sulfate, or copper pyrophosphate. The copper composition may also be a copper amino acid complex, such as copper aspartate, copper glutamate, copper glycinate, copper histidinate, copper lysinate, copper methionate, copper tyrosinate, or copper complexed to a mixture of amino acids. The copper composition may also be a copper carboxylic acid complex, such as copper ascorbate, copper citrate, copper formate, copper fumarate, copper gluconate, copper glutarate, copper malate, copper orotate, copper picolinate, copper sebacate, copper succinate, or copper tartrate. The copper may also be complexed with nucleotides, polysaccharides, or proteinates. The copper composition may also be a copper chelate, such as copper EDTA.

In yet another embodiment, the metal source may comprise iron. The iron composition may be an iron salt, such as iron chloride, iron nitrate, iron oxide, iron phosphate, iron pyrophosphate, or iron sulfate. The iron composition may also be an iron amino acid complex, such as iron aspartate, iron argininate, iron bisglycinate, iron glycinate, iron histidinate, iron ketoglutarate, iron lysinate, iron methionate, or iron complexed to a mixture of amino acids. The iron composition may also be an iron carboxylic acid complex, such as iron ascorbate, iron citrate, iron gluconate, iron lactate, iron malate, iron oxalate, iron orotate, iron pantothenate, iron picolinate, or iron succinate. The iron composition may also be another organic complex, such as iron ethanolamine phosphate, iron glucoheptonate, or iron peptonate. The iron may also be complexed to nucleotides, polysaccharides, or proteinates. The iron composition may also be an iron chelate, such as iron DTPA, iron EDDHA, or iron EDTA.

In still another embodiment, the metal source may comprise manganese. The manganese composition may be a manganese salt, such as manganese acetate, manganese carbonate, manganese chloride, manganese dioxide, manganese nitrate, manganous oxide, or manganese sulfate. The composition may also be a manganese amino acid complex, such as manganese argininate, manganese aspartate, manganese cysteinate, manganese glycinate, manganese histidinate, manganese ketoglutarate, manganese methionate, or manganese complexed to a mixture of amino acids. The manganese composition may also be a carboxylic acid complex, such as manganese ascorbate, manganese formate, manganese fumarate, manganese lactate, manganese malate, manganese orotate, or manganese picolinate. The manganese composition may also another organic complex, such as manganese ethanolamine phosphate or manganese peptonate. The manganese may also be complexed to nucleotides, polysaccharides, or proteinates. The manganese composition may also be a manganese chelate, such as manganese EDTA.

In yet another embodiment, the mineral source may comprise selenium. The selenium composition may be inorganic, such as selenium oxychloride, selenium sulfide, sodium selenite, or selenous acid. The selenium composition may also be a selenium amino acid complex or a selenium containing amino acid. Examples include, selenium glycinate, selenium lysinate, selenocysteine, or selenomethionine. The selenium composition may also be a selenium carboxylic acid complex, such as selenium ascorbate, selenium citrate, selenium fumarate, selenium malate, selenium picolinate, or selenium succinate. The selenium composition may also be another organic complex, such as selenium ethanolamine phosphate or selenium peptonate.

In an exemplary embodiment, the metal source may be a metal salt or a metal chelate comprising at least one hydroxyl analog of methionine corresponding to formula (I):

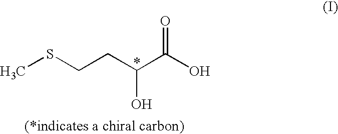

The compound having formula (I) is 2-hydroxy-4(methylthio)butanoic acid (commonly known as “HMTBA” and sold by Novus International, St. Louis, Mo. under the trade name Alimet®). Representative salts of HMTBA, in addition to the ones described below, include the ammonium salt, the stoichiometric and hyperstoichiometric alkaline earth metal salts (e.g., magnesium and calcium), and the stoichiometric and hyperstoichiometric alkali metal salts (e.g., lithium, sodium, and potassium).

Alternatively, the metal source may be a metal chelate comprising one or more ligand compounds having formula (I) together with one or more metal ions. Irrespective of the embodiment, suitable non-limiting examples of metal ions include zinc ions, copper ions, manganese ions, magnesium ions, iron ions, chromium ions, selenium ions, cobalt ions, potassium ions, and calcium ions. In one embodiment, the metal ion is divalent. Examples of divalent metal ions (i.e., ions having a net charge of 2⁺) include copper ions, zinc, ions, manganese ions, magnesium ions, calcium ions, cobalt ions and iron ions. In another embodiment, the metal ion is zinc. In yet another embodiment, the metal ion is copper. In still another embodiment, the metal ion is iron. In a further embodiment, the metal ion is calcium. In one exemplary embodiment, the metal chelate is HMTBA-Mn. In a further exemplary embodiment, the metal chelate is HMTBA-Cu. In an alternative exemplary embodiment, the metal chelate is HMTBA-Zn. In still another exemplary embodiment, the metal chelate is HMTBA-Fe. As will be appreciated by a skilled artisan, the ratio of ligands to metal ions forming a metal chelate compound can and will vary. Generally speaking, a suitable ratio of ligand to metal ion is from about 1:1 to about 3:1 or higher. In another embodiment, the ratio of ligand to metal ion is from about 1.5:1 to about 2.5:1. Of course within a given mixture of metal chelate compounds, the mixture will include compounds having different ratios of ligand to metal ion. For example, a composition of metal chelate compounds may have species with ratios of ligand to metal ion that include 1:1, 1.5:1, 2:1, 2.5:1, and 3:1.

In another exemplary embodiment, the metal source may be a metal complex comprising one or more glycine molecules together with one or more metal ions. Suitable non-limiting examples of metal ions include zinc ions, copper ions, manganese ions, magnesium ions, iron ions, chromium ions, selenium ions, cobalt ions, potassium ions, and calcium ions. In one embodiment, the metal ion comprises zinc and copper. In another embodiment, the metal ion is copper. In a preferred embodiment, the metal ion is zinc.

(b) Methods for Administration

The metal source may be administered to an animal by a variety of suitable methods generally known in the art. For example, the metal source may be mixed directly with the animal feed, either as a dry or a liquid composition. The metal composition may be mixed with water, a saline mixture, or a liquid feed composition. The metal composition may be administered through gavage, as a bolus, or as a direct injection. The injection may be subcutaneous, intramuscular, or intravenous.

The time course of administration can and will vary depending upon the application. The metal source may be administered in one dose. The metal source may be administered in multiple doses. Or the metal source may be administered continuously (e.g., in the feed or water).

As will be appreciated by a skilled artisan, the amount of metal in the metal source to be administered to an animal can and will vary depending on the type of animal, its age, gender, and nutritional status. Generally speaking, the amount of metal source administered to the animal will typically contain enough metal such that the metal source causes a detectable modulation in the expression of a biomarker.

### (II) Collecting a Sample from an Animal

After administering the metal source to an animal, typically a biological sample is collected from the animal and the level of biomarker expression in the sample may be determined. In general, the biological sample is preferably collected from the animal during the time when the biomarker's level of expression is modulated in response to the metal source. For example, depending upon the biomarker, the biological sample may be collected from about 30 minutes to about two weeks after the animal is administered the metal source. In another embodiment, the biological sample is collected from about four hours to about 5 days after the animal is administered the metal source. In an additional embodiment, the biological sample is collected from about 12 hours to about 3 days after the animal is administered the metal source. As will be appreciated by a skilled artisan, the number of biological samples collected can and will vary depending upon particular application. In some embodiments, collection of a single biological sample may be sufficient. In other embodiments, collection of multiple biological samples may be desirable.

The biological sample may be a cell, a collection of cells, a cell extract, a tissue, a piece of tissue, a tissue extract, a bodily fluid, or a biopsy sample. The sample may be derived from the gastrointestinal tract. Suitable gastrointestinal samples include, but are not limited to, the small intestine (duodenum, jejunum, ileum), or the large intestine (cecum, colon, rectum, anal canal). The gastrointestinal samples may be pieces of tissue or they may be mucosal scrapings. Samples may also be derived from, but not limited to, heart, brain, placenta, liver, skeletal muscle, crop, stomach, kidney, pancreas, spleen, thymus, prostate, testis, and uterus. Samples may also be derived from bodily fluids, including saliva, sputum, urine, lymph, blood, or blood products, such as plasma, serum and white blood cells. Samples may also be obtained by biopsy, including needle biopsy or incisional biopsy. In one embodiment, the sample may be a blood sample, preferably a sample of white blood cells. In another embodiment, the sample may be a section of jejunum. In yet another embodiment, the sample may be jejunal mucosal scrapings.

### (III) Detection of Metal Responsive Biomarkers

(a) Suitable Metal Responsive Biomarkers

Several metal responsive biomarkers are suitable for use in the method of the invention. Generally speaking, a metal responsive biomarker is suitable to the extent that the biomarker's level of expression is modulated in response to the metal source administered to an animal and to the extent that the biomarker can be detected and/or quantified in a biological sample. As such, there are several genes whose transcription is regulated by metals, and these may serve as good biomarkers of metal status or bioavailability. By way of example, Table A lists several genes whose transcription is regulated (positively or negatively) by metals. Table A lists suitable examples of metal responsive genes, and as appreciated by one skilled in the art, the list is not exhaustive. As shown in the table, some genes respond to multiple metals. For example, the expression of metallothionein (MT) is induced by zinc, but its expression also increases in response to cadmium, chromium, copper, iron, manganese, or selenium. Similarly, the expression of superoxide dismutases is induced by zinc, copper, manganese, iron and nickel. The table also lists some genes that respond primarily to one metal, such as copper, iron, selenium, or zinc. In one embodiment, the biomarker may be metal-regulatory transcription factor 1 (MTF1). In another embodiment, the biomarker may be iron response element binding protein 1 (IRE-BP1). In yet another embodiment, the biomarker may be a superoxide dismutase (SOD). In still another embodiment, the biomarker may be cytochrome c oxidase chaperone (COX17). In yet another embodiment, the biomarker may be ceruloplasmin. In an alternate embodiment, the biomarker may be a zinc transport protein. In a preferred embodiment, the biomarker may be metallothionein (MT).

TABLE A
Metal Responsive Genes

| | | | |
|---|---|---|---|
| | | Chicken | Cattle |
| Gene Name | Gene Annotation | Accession No. | Accession No. |

Multiple Metal-Responsive Genes

| | | | |
|---|---|---|---|
| MT | metallothionein | NM_205275 | M79677 |
| MT2 | metallothionein 2 | | XM_586929 |
| SOD1 | superoxide | NM_205064 | NM_174615 |
| | dismutase 1, | | |
| | soluble | | |
| SOD2 | superoxide | NM_204211 | NM_201527 |
| | dismutase 2, | | |
| | mitochondrial | | |

Copper (Cu)-Responsive Genes

| | | | |
|---|---|---|---|
| ATOX1 | antioxidant protein 1 | | XM_877874 |
| | homolog (yeast) | | |
| ATP7A | ATPase, Cu | XM_420308 | |
| (Menkes) | transporter, | | |
| | (Menkes syndrome) | | |
| CCS | copper chaperone | | NM_001046187 |
| | for superoxide | | |
| | dismutase | | |
| COX17 | cytochrome c | XM_425526 | XM_872678 |
| | oxidase chaperone | | |
| SLC31A1 | high-affinity copper | member 2: | member 1; |
| | uptake protein, | XM_423492 | XM_597183 |
| | solute carrier family | | |
| | 31 | | |
| cerulo- | also called | XP_417192 | isoform 1: |
| plasmin | ferroxidase | | XP_552003 |

Iron (Fe)-Responsive Genes

| | | | |
|---|---|---|---|
| ABCB | ATP-binding | | XM_590317 |
| | cassette transporter | | |
| FRDA | frataxin | XM_424827 | |
| TF | transferrin | NM_205304 | |
| HMOX2 | heme oxygenase 2 | NM_414960 | NM_001035087 |
| IRE-BP1 | iron response | | NM_001075591 |
| | element binding | | |
| | protein 1 | | |
| FTL | ferritin, light chain | | XM_872471 |
| FTH | ferritin, heavy chain | NM_205086 | |
| LTF | lactotransferrin | | NM_180998 |

Selenium (Se)-Responsive Genes

| | | | |
|---|---|---|---|
| SEP | selenoprotein P | XM_422432 | NM_001046048 |
| GPX3 | glutathione | | NM_174077 |
| | peroxidase 3 | | |
| GPX5 | glutathione | | NM_001025335 |
| | peroxidase 5 | | |
| SPS2 | selenophosphate | XM_424246 | XM_865819 |
| | synthetase 2 | | |

Zinc (Zn)-Responsive Genes

| | | | |
|---|---|---|---|
| SLC30 | solute carrier family | member 5: | member 4: |
| ZnT exporter | 30 (zinc transporter) | NM_001031419 | XM_606905 |
| family | | | |
| SLC39 | solute carrier family | member 9: | member 4: |
| ZIP importer | 39 (zinc transporter) | NM_001007933 | NM_001046067 |
| family | | | |
| MTF1 | metal-regulatory | NM_001031495 | NM_001035080 |
| | transcription | | |
| | factor 1 | | |

Other Metal Responsive Genes

| | | | |
|---|---|---|---|
| CAT | catalase | NM_001031215 | NM_0010345386 |
| GSTA1 | glutathione-S- | NM_001001777 | |
| | transferase, A1 | | |
| GSTA2 | glutathione-S- | | NM_177515 |
| | transferase, A2 | | |
| Hsp70 | heat shock protein | NM_001006688 | NM_174550 |
| | 70 | | |
| Hsp27 | heat shock protein | NM_001001527 | NM_001025569 |
| | 27 | | |

(b) Detection Methods

Measuring the expression of the biomarker may be accomplished by a variety of techniques that are well known in the art. Expression may be monitored by detecting the mRNA or protein products of the biomarker genes. RNA or protein may be isolated from samples of interest using techniques well known in the art and disclosed in standard molecular biology reference books, such as Ausubel et al., (2003) Current Protocols in Molecular Biology, John Wiley & Sons, New York, N.Y.

(i) Detecting RNA

Detection of the RNA products of the biomarker may be accomplished by a variety of methods. Some methods are quantitative and allow estimation of the original levels of RNA in the cells or tissues of interest, whereas other methods are merely qualitative. Additional information regarding the methods presented below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. A person skilled in the art will know which parameters may be manipulated to optimize detection of the mRNA of interest.

Quantitative real-time PCR (QRT-PCR) may be used to measure the differential expression of a biomarker under different conditions. In QRT-PCR, the RNA template is generally reverse transcribed into cDNA, which is then amplified via a PCR reaction. The amount of PCR product generated is followed cycle-by-cycle in real time, which ultimately allows for determination of the initial concentrations of mRNA or cDNA in the sample. Quantitation may be relative or absolute. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified. A non limiting example of a fluorescent reporter probe is a TaqMan®) probe (Applied Biosystems, Foster City, Calif.). The fluorescent reporter probe fluoresces when the quencher is removed during the PCR extension cycle. Fluorescence values are recorded during each cycle and represent the amount of product amplified to that point in the amplification reaction. To minimize errors and reduce any sample-to-sample variation, QRT-PCR is typically performed using an external and/or an internal standard. The ideal standard is expressed at a constant level among different tissues, and is unaffected by the experimental treatment. Suitable internal standards include, but are not limited to, mRNAs for the housekeeping genes glyceraldehyde-3-phosphate-dehydrogenase (GAPDH), beta-actin, or 18S rRNA.

Reverse-transcriptase PCR (RT-PCR) may also be used to measure the expression of a biomarker. As above, the RNA template is reverse transcribed into cDNA, which is then amplified via a typical PCR reaction. After a set number of cycles the amplified DNA products are typically separated by gel electrophoresis. Comparison of the relative amount of PCR product amplified in the different samples will reveal whether the biomarker is differentially expressed.

Expression of a biomarker may also be measured using a nucleic acid microarray. In this method, single-stranded nucleic acids (e.g., cDNAs, oligonucleotides) are plated, or arrayed, on a microchip substrate. The arrayed sequences are then hybridized with specific DNA probes generated from the samples of interest. Fluorescently labeled cDNA probes may be generated through incorporation of fluorescently labeled deoxynucleotides by reverse transcription of RNA extracted from the samples of interest. The probes are hybridized to the immobilized nucleic acids on the microchip under highly stringent conditions. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA (e.g., control and treatment) are hybridized pairwise to the array. The relative abundance of the transcripts from the two sources corresponding to each specified biomarker is thus determined simultaneously. Microarray analysis may be performed by commercially available equipment, following manufacturer's protocols, such as by using the Affymetrix GenChip technology, or Incyte's microarray technology.

Expression of a biomarker may also be measured using Luminex microspheres, in which molecular reactions take place on the surface of microscopic polystyrene beads. The beads are internally color-coded with fluorescent dyes, such that each bead has a unique spectral signature (of which there are up to 100). The surface of each bead is tagged with a specific oligonucleotide that can bind the target (i.e., mRNA) of interest. The target, in turn, is often attached to a reporter, which is also fluorescently tagged. Hence, there are two sources of color, one from the bead and the other from the reporter molecule. The small size/surface area of the beads and the three dimensional exposure to the targets allows for nearly solution-phase kinetics during the binding reaction. The captured targets are detected by high-tech fluidics based upon flow cytometry in which lasers excite the internal dyes that identify each bead and also any reporter dye captured during the assay.

Differential expression of a biomarker may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe. After washing to remove the non-specifically bound probe, the hybridized labeled species are detected using techniques well known in the art. The probe may be labeled with a radioactive element, a chemical that fluoresces when exposed to ultraviolet light, a tag that is detected with an antibody, or an enzyme that catalyses the formation of a colored or a fluorescent product. A comparison of the relative amounts of RNA detected in the different samples will reveal whether the expression of the biomarker is changed.

Nuclease protection assays may also be used to monitor the differential expression of a biomarker. In nuclease protection assays, an antisense probe hybridizes in solution to an RNA sample. The antisense probe may be labeled with an isotope, a fluorophore, an enzyme, or another tag. Following hybridization, nucleases are added to degrade the single-stranded, unhybridized probe and RNA. An acrylamide gel is used to separate the remaining protected double-stranded fragments, which are then detected using techniques well known in the art. Again, qualitative differences in expression may be detected.

Differential expression of a biomarker may also be measured using in situ hybridization. This type of hybridization uses a labeled antisense probe to localize a particular mRNA in cells of a tissue section. The hybridization and washing steps are generally performed under highly stringent conditions. The probe may be labeled with a fluorophore or a small tag (such as biotin or digoxigenin) that may be detected by another protein or antibody, such that the labeled hybrid may be visualized under a microscope. The relative abundance and location of the transcripts in the cell may be visualized.

(ii) Detecting Protein

Detection of the protein products of the biomarker may be accomplished by several different techniques, many of which are antibody-based. Additional information regarding the methods discussed below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the protein of interest.

An enzyme-linked immunosorbent assay or ELISA may be used to detect and quantitate protein levels. Many types of ELISA assays exist. One method, called a “sandwich ELISA” is particularly suited to detect the presence of a protein biomarker. In this method, a known quantity of an antibody specific to the biomarker is coated on the wells of a microtiter plate. The sample potentially containing the biomarker is then applied to the plate and incubated, and then the plate is washed to remove unbound sample. A second antibody specific for the biomarker is applied. This second antibody is generally conjugated to an enzyme, such as horseradish peroxidase or alkaline phosphatase, or to another marker, any of which will generate colorimetric, fluorescent, or chemiluminescent products for detection. The plate is washed to remove unbound antibody. The remaining antibody is detected and quantified as an indicator of biomarker concentration in the sample. One skilled in the art will recognize that other ELISA methods also exist which may be suitable.

Proteins of interest may also be detected using Luminex beads as described above in section (III)(b)(i), except that antibodies are attached to the surface of the beads.

Relative protein levels may also be measured by Western blotting. Western blotting generally comprises preparing protein samples, using gel electrophoresis to separate the denatured proteins by mass, and probing the blot with antibodies specific to the protein of interest. Detection and quantitation is usually accomplished using two antibodies, the second of which is conjugated to an enzyme for detection or another reporter molecule. Methods used to detect differences in protein levels include calorimetric detection, chemiluminescent detection, fluorescent detection, and radioactive detection.

Measurement of protein levels may also be performed using a protein microarray or an antibody microarray. In one aspect of this method, antibodies against the biomarker proteins of interest are covalently attached to the surface of a microarray or biochip. A sample potentially containing the biomarker proteins is contacted with the array of antibodies such that biomarker proteins bind the antibodies, the unbound proteins are washed off, and the antibody/antigen complexes are detected, generally detected via fluorescent tags on the antibody.

Relative protein levels may also be assessed by immunohistochemistry, in which a biomarker protein is localized in cells of a tissue section by its interaction with a specific antibody. The antigen/antibody complex may be detected and visualized by a variety of methods. The detection antibody may be tagged with a fluorophore, or it may be conjugated to an enzyme that catalyzes the production of a detectable product. The labeled complex is typically visualized under a microscope.

### (IV) Applications

The method of the invention may be utilized for a variety of suitable applications. In one embodiment, the method may be used to determine relative metal bioavailability of a metal source administered to an animal or to a group of animals. In this context of this invention, the phrase “relative metal bioavailability” is used in its broadest sense to mean the approximate degree to which a metal is absorbed and available to the animal. In the method, the animal or group of animals is typically administered a metal source, a biological sample is then collected from the animal (or group of animals), and the level of expression of a metal responsive biomarker in a sample is detected. The same series of steps is also performed in a control animal or group of animals. Typically, a “control animal” is an animal of the same species, of similar age and body weight, and preferentially the same sex as the test animal. In this particular method, the control animal is fed the same diet as the test animal, but is not administered the metal source. In this manner, the relative bioavailability of the metal source in the test animal may be determined by comparing the level of expression of the biomarker in samples collected from the test animal and control animal. In general, a difference in the level of expression between the two samples indicates the relative bioavailability of the metal source in the animal. In another alternative embodiment of this method, the control may be a sample obtained from the test animal prior to administering the metal source. In this embodiment, the relative bioavailability of the metal source in the test animal may be determined by comparing the level of expression of the biomarker in samples collected from the test animal before and after the animal has been administered the metal source. A difference in the level of expression between the two samples indicates the relative bioavailability of the metal source in the animal.

A further aspect of the invention encompasses a method for comparing the relative metal bioavailability between different metal sources. The method comprises administering a first metal source to a first animal (or group of animals) and a second metal source to a second animal (or group of animals); detecting the level of expression of a metal responsive biomarker present in a first sample obtained from the first animal and in a second sample obtained from the second animal; and comparing the level of biomarker expression in each sample. Typically, a difference in the level of expression of the biomarker indicates that the first and second metal sources have different relative metal bioavailability. Depending upon the embodiment, the method may be utilized to test the relative bioavailability of several different metal sources. In one embodiment, the relative metal bioavailability of two different metal sources may be compared. In still another embodiment, the relative metal bioavailability of three different metal sources may be compared. In yet another embodiment, the relative metal bioavailability of four different metal sources may be compared. In an additional embodiment, the relative metal bioavailability of five different metal sources may be compared. In a further embodiment, the relative metal bioavailability of greater than five different metal sources may be compared.

The method for comparing the relative metal bioavailability between different metal sources has several additional embodiments. The different metal sources may be provided at the same levels. Alternatively, the different metal sources may be provided at different levels. For example, the bioavailability of metal source 1 provided at 30 ppm metal may be compared to the bioavailability of metal source 2 provided at 75 ppm metal. In another embodiment, the bioavailability of a metal provided as a supplement may be compared to the bioavailability of a metal provided naturally in the diet (i.e., as part of a corn-soy diet, a hay diet, a semi-purified diet, or any other diet).

Yet another aspect of the invention provides a method for determining the relative nutritional status of a metal in an animal or group of animals. As used in the context of the present invention, the phrase “nutritional status” refers to the relative amount of a metal present in the animal. If the amount is too low, the animal may have a metal deficiency and if the amount is grossly elevated, depending upon the metal, the animal may develop toxicity. To determine the nutritional status of a metal, the method comprises detecting the level of metal responsive biomarker expression in a sample obtained from the animal and in a control animal sample, and comparing the level of biomarker expression in each sample. By way of example, when the zinc or copper status is determined, and MT is the biomarker, a lower level of metallothionein mRNA in the sample from the test animal versus the control animal sample indicates that the animal may have a zinc or copper deficiency.

Utilizing the various methods for determining relative metal bioavailability and nutritional status provided by the invention, in certain embodiments, it may be possible to optimize the mineral content of a diet for an animal or a group of animals. In this context, the phrase “optimize” generally means determining an amount of mineral that results in optimum growth and efficiency, and the ability of the animal to achieve a variety of biological markers, including, but not limited to, immune function, reproductive function, tissue strength, bone development or strength, hoof health, and proper enzymatic activity. Typically, to optimize the mineral content for an animal diet several different amounts of mineral are fed to the animal and the relative mineral bioavailability for each amount is determined according to the methods of the invention.

While the guidelines for the proper optimal mineral amount for a given animal species established by The National Research Council may be used as a starting point, one skilled in the art would recognize that the optimal amount of a mineral to administer to an animal is typically higher than the recommended guidelines. By way of non-limiting example, however, the following NRC-guidelines may be consulted to determine a starting point for optimization of a mineral amount: Nutrient Requirements of Poultry: Ninth Revised Edition, (1994) (National Acad. Press); Nutrient Requirements of Swine: 10th Revised Edition (1998) (National Acad. Press); Nutrient Requirements of Beef Cattle: Seventh Revised Edition: (2000) (National Acad. Press); Nutrient Requirements of Dairy Cattle: Seventh Revised Edition: (2001) (National Acad. Press); Nutrient Requirements of Dogs and Cats (2003) (National Acad. Press); Effect of Environment on Nutrient Requirements of Domestic Animals (1981) (National Acad. Press); Nutrient Requirements of Goats: Angora, Dairy, and Meat Goats in Temperate and Tropical Countries (1981) (National Acad. Press); Nutrient Requirements of Sheep, Sixth Revised Edition, 1985 (1985) (National Acad. Press), and the like). As an example, the Zinc requirements suggested by the National Research Council are briefly summarized in Table B.

TABLE B
NRC Dietary Recommendations

| Class of Animal | Zinc Requirement in Diet (ppm) |
|---|---|
| Chicken | 40 |
| Turkey | 40–70 |
| Beef Cattle | 30 |
| Dairy Cattle | 23–63 |
| Swine | 50–100 |
| Horse | 40 |
| Sheep | 20–33 |
| Goat | 45–75 |

### DEFINITIONS

The term “expression,” as used herein, refers to the conversion of the DNA sequence information into messenger RNA (mRNA), ribosomal RNA (rRNA), or protein. Expression may be monitored by measuring the levels of full-length mRNA, mRNA fragments, full-length rRNA, rRNA fragments, full-length protein, or protein fragments.

The term “hybridization,” as used herein, refers to the process of binding, annealing, or base-pairing between two single-stranded nucleic acids. The “stringency of hybridization” is determined by the conditions of temperature and ionic strength. Nucleic acid hybrid stability is expressed as the melting temperature or Tm, which is the temperature at which the hybrid is 50% denatured under defined conditions. Equations have been derived to estimate the Tm of a given hybrid; the equations take into account the G+C content of the nucleic acid, the length of the hybridization probe, etc. (e.g., Sambrook et al, 1989, chapter 9). To maximize the rate of annealing of the probe with its target, hybridizations are generally carried out in solutions of high ionic strength (6×SSC or 6×SSPE) at a temperature that is about 20-25° C. below the Tm. If the sequences to be hybridized are not identical, then the hybridization temperature is reduced 1-1.5° C. for every 1% of mismatch. In general, the washing conditions should be as stringent as possible (i.e., low ionic strength at a temperature about 12-20° C. below the calculated Tm). As an example, highly stringent conditions typically involve hybridizing at 68° C. in 6×SSC/5× Denhardt's solution/1.0% SDS and washing in 0.2×SSC/0.1% SDS at 65° C. The optimal hybridization conditions generally differ between hybridizations performed in solution and hybridizations using immobilized nucleic acids. One skilled in the art will appreciate which parameters to manipulate to optimize hybridization.

As various changes could be made in the above compounds, methods, and products without departing from the scope of the invention, it is intended that all matter contained in the above description and in the examples given below, shall be interpreted as illustrative and not in a limiting sense.

### EXAMPLES

The following examples illustrate several embodiments of the invention.

### Example 1

### Induction of Metallothionein by Zinc in Chickens

This experiment was performed to determine whether metallothionein would be a useful biomarker to determine the bioavailability of zinc. The levels of mRNA were measured by quantitative real-time RCR (QRT-PCR).

Zinc treatment and sample collection. Upon hatching, chicks were placed on a moderately Zn-deficient starter diet. On day 13, they were switched to two different test diets: some were fed a low zinc diet with no zinc supplements (the diet contained 34 ppm zinc in the basal feedstuffs) and the rest were fed a high zinc diet comprising 34 ppm zinc in the feedstuffs and 64 ppm zinc from Zn(HMTBA)₂ for a total of 98 ppm zinc. On day 29, a chicken from each group was sacrificed and the following tissues were collected: jejunum (small intestine), jejunum mucosal scrapings, liver, and kidney. The lumen of a 2-3 inch long jejunum section was rinsed with 3-4 ml of cold saline. The jejunum was cut open lengthwise and placed mucosa-side up on a clean cutting board. One piece of jejunum, no greater than 0.5 cm in any direction, was placed into a microcentrifuge tube containing 0.9 ml of RNAlater solution, and stored according to the manufacturer's instructions (Ambion, Austin Tex.). The jejunum section's remaining mucosal surface was scraped with a glass slide. Cell scrapings were collected and placed into 0.9 ml RNAlater. The other tissues were stored in RNAlater. RNA was isolated using the High Pure RNATissue Prep Kit (Roche Applied Science, Cat No 12033674001) following the manufacturer's instructions. The concentration of RNA was determined using a spectrophotometer, and the concentration of RNA was adjusted to a maximum of 0.5 mg/ml by dilution with elution buffer. The RNA samples were either used immediately, or frozen on dry ice and stored at −80° C. until use.

Reverse Transcriptase and Quantitative PCR. First strand cDNA synthesis was performed using the Transcriptor Reverse Transcriptase® (Roche Applied Science, Indianapolis, Ind., catalog #04379012001), using the instructions supplied by the manufacturer. Each first-strand synthesis reaction contained 0.5 μg of total RNA. The reverse transcription was performed in a thermocycler using the following program: a brief, 90 sec denaturation step at 65° C.; annealing for 30 sec at 40° C.; 30 min cDNA synthesis at 55° C.; and enzyme inactivation by 5 min denaturation at 85° C. The reaction product was concentrated by ethanol precipitation, rinsed and dried. The sample was then resuspended in 400 μl of nuclease-free water and A260/A280 was measured to ensure that cDNA concentration in each sample would fall within the range of concentration usable by the QRT-PCR assay.

MT mRNA and 18S rRNA levels were measured by QRT-PCR on a Roche LightCycler®, using the LightCycler® FastStart DNA Master HybProbe Kit (Roche Molecular Systems, Inc, Alameda, Calif., catalog #300324) using 0.5 μM of each primer and 0.2 μM of each TaqMan probe, as shown in Table 1. The parameters of the assay were 42 cycles of 10 seconds at 95° C. and 40 seconds at 60° C.

TABLE 1

Primers used for QRT-PCR in chicken.

| | | SEQ |
|---|---|---|
| | | ID |
| Primer Name | Sequence | NO |
| cMT Forward Primer | 5′-CCTGTGCTGGGTCGT-3′ | 1 |
| cMT Reverse Primer | 5′-TGCTGGCCGGTTCCT-3′ | 2 |
| cMT Taqman Probe | 5′-FAM-TGCTGCTCCTGCTGCC- | 3 |
| | BHQ1-3′ | |
| c18S Forward Primer | 5′-GGCCGCCGGAATACT-3′ | 4 |
| c18S Reverse Primer | 5′-TCTTGCGCCGGTCC-3′ | 5 |
| c18S Taqman Probe | 5′-FAM- | 6 |
| | CCATGATTAAGAGGGACGGCC-BHQ1- | |
| | 3′ | |

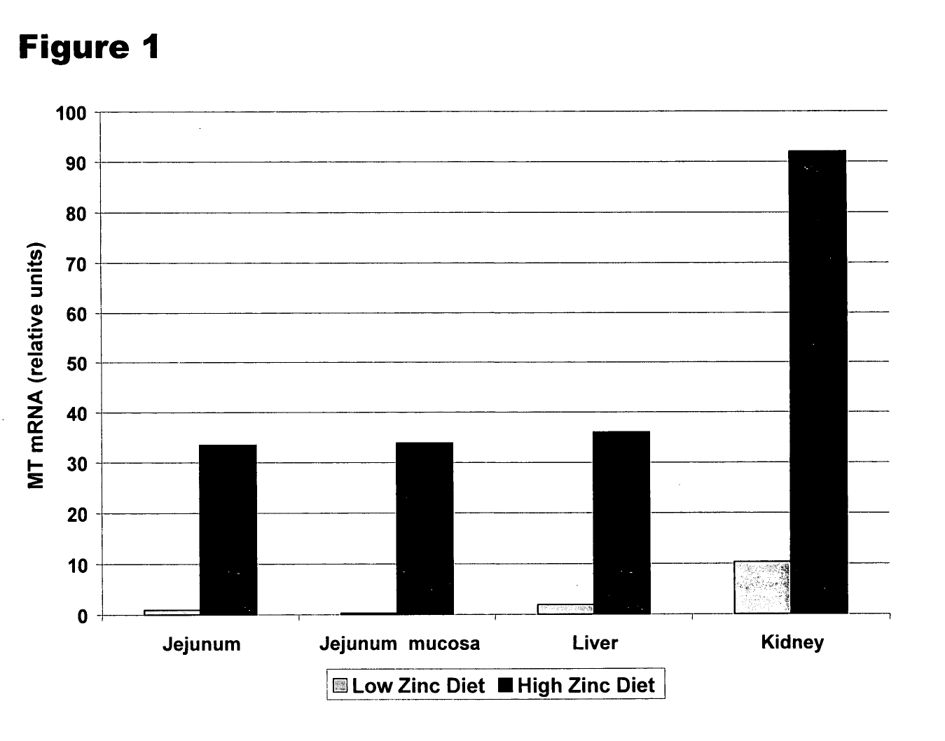

Results. QRT-PCR revealed that MT mRNA levels were increased in the presence of high zinc relative to low zinc, whereas 18S rRNA levels were not affected by zinc concentration. Therefore, MT mRNA expression was normalized to 18S rRNA levels in each sample. FIG. 1 presents MT mRNA expression in the different tissues by tissue and by treatment. Each bar represents MT expression relative to MT expression in the low zinc diet jejunum sample (Low Zinc Diet jejunum MT expression=1). The fold inductions of MT mRNA shown in this experiment were: 34-fold in jejunum, 125-fold in jejunum mucosal scrapings, 19-fold in liver, and 9-fold in kidney. These data reveal that MT expression is induced by zinc.

### Example 2

### Time Course of Zinc-induced Expression of Metallothionein in Chickens

The purpose of this experiment was to establish the time course of up-regulation of the metallothionein gene by zinc.

A total of 54 chickens were fed a low zinc/low copper milo starter diet for 10 days. On day 11 (t=Day 0), the birds were split into two groups: 18 were fed a corn-soy diet supplemented with 25 ppm copper oxide and no zinc (“Cu”) and 36 were fed a corn-soy diet supplemented with 25 ppm copper oxide and 70 ppm zinc from Zn(HMTBA)₂ (“Cu+Zn(HMTBA)₂”). On days 13 (t=Day 2), 16 (t=Day 5) and 19 (t=Day 8), three “Cu” and three “Cu+Zn(HMTBA)₂” birds per day were weighed and sacrificed. The mid-jejunum was collected from each bird. The lumen was rinsed with 3-4 ml of cold saline, and each sample was divided into two pieces, with each piece being no larger than 0.5 cm in any direction. The two pieces from each bird were placed in separate microcentrifuge tubes containing 0.9 ml of RNAlater solution.

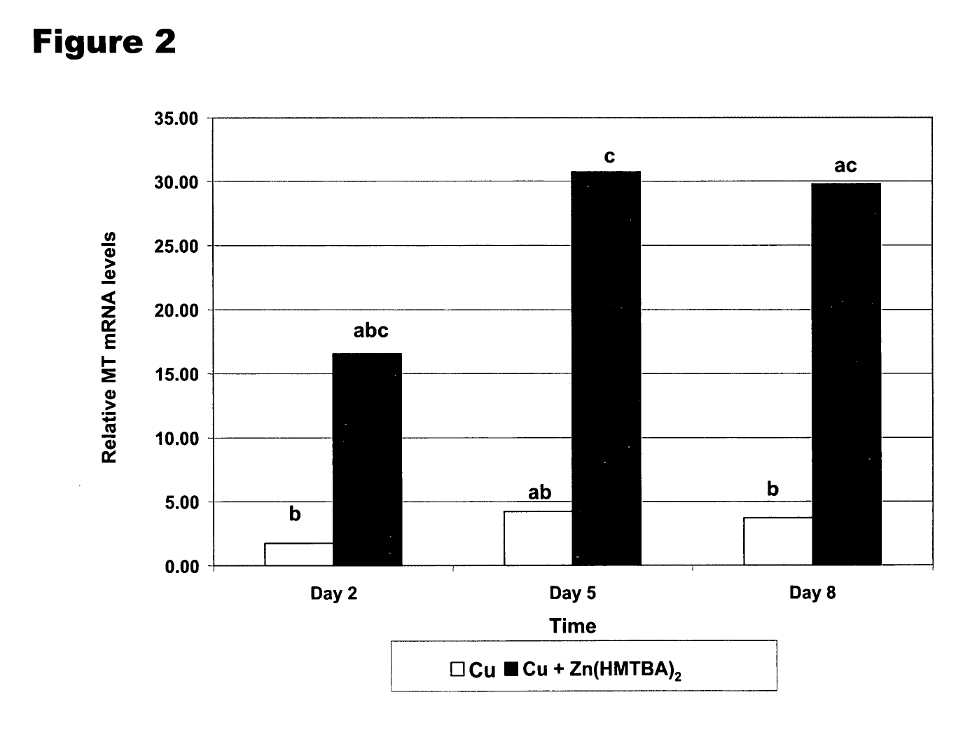

RNA was isolated (RNA from the two pieces from each bird were pooled), and 18S-normalized MT mRNA expression levels were measured by QRT-PCR as described in Example 1. The results are presented in FIG. 2. The data are expressed as MT mRNA expression relative to the low zinc jejunum reference sample from Example 1. Significant differences in MT expression are seen between treatments on days 5 and 8. Overall, Zn(HMTBA)₂ increased MT expression with a P=0.0077.

### Example 3

### Bioavailability of Different Zinc Compositions in Chickens

The bioavailability of various zinc compositions was compared by measuring the levels of MT mRNA using QRT-PCR.

Chickens were fed a low zinc pre-feed diet for 20 days. On day 21, they were switched to a corn-soy diet supplemented with one of the following: 1) unsupplemented for zinc (control); 2) 70 ppm zinc from ZnOxide; 3) 70 ppm zinc from a zinc amino acid complex (Zn AAC); 4) 70 ppm zinc from Zn(HMTBA)₂. On day 23, six birds from each treatment were weighed, sacrificed, and jejunum samples were collected. The lumen was rinsed with 3-4 ml of cold saline, the jejunum was cut open lengthwise, and the mucosal surface was scraped with a glass slide. Cell scrapings were collected into 0.9 ml of RNAlater. RNA was isolated as described in Example 1. QRT-PCR was performed as described in Example 1, and 18S-normalized MT mRNA expression was determined.

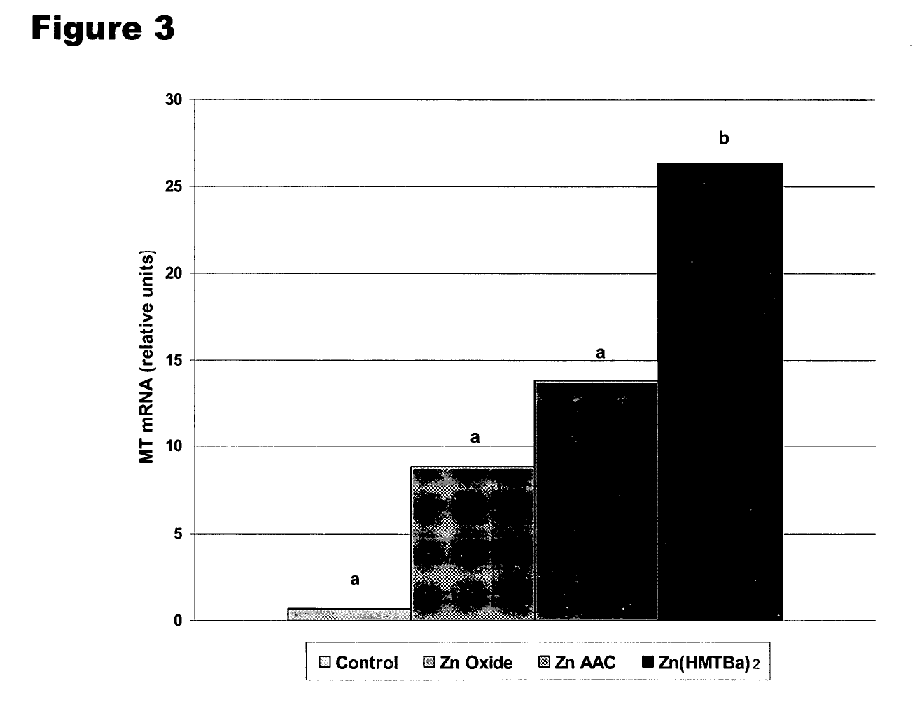

As shown in FIG. 3, the levels of MT induced by Zn(HMTBA)₂ were significantly greater than those induced by ZnOxide or the Zn AAC. These data indicate that Zn(HMTBA)₂ is more bioavailable than ZnOxide or the Zn AAC. MT expression in the birds fed ZnOxide and the Zn AAC was not significantly greater than MT expression in the unsupplemented birds.

### Example 4

### Bioavailability of Different Metal Compositions in Dairy Cattle

For one week, 20 lactating multiparous dairy cows were fed a basal diet that was formulated to approximate NRC (2001) trace mineral requirements for lactating cows. The diets were formulated to contain 47 ppm of zinc, 11 ppm of copper, and 43 ppm of manganese. At the end of one week (t=Week 0), liver biopsies were taken from each cow. The samples were stored in RNAlater.

Then, all of the cows were switched to a high mineral diet that was the basal diet supplemented with one of two metal compositions (10 cows per treatment). The first metal premixture (Mix 1) delivered 320 mg zinc as Zn(HMTBA)₂, 150 mg copper from Cu(HMTBA)₂, and 130 mg manganese from Mn(HMTBA)₂ per head per day. The second metal premixture (Mix 2) delivered the same amount of additional Zn, Cu and Mn as specific amino acid complexes. After one week on the higher mineral diet (t=Week 1), liver biopsies were taken, and the samples were stored in RNAlater.

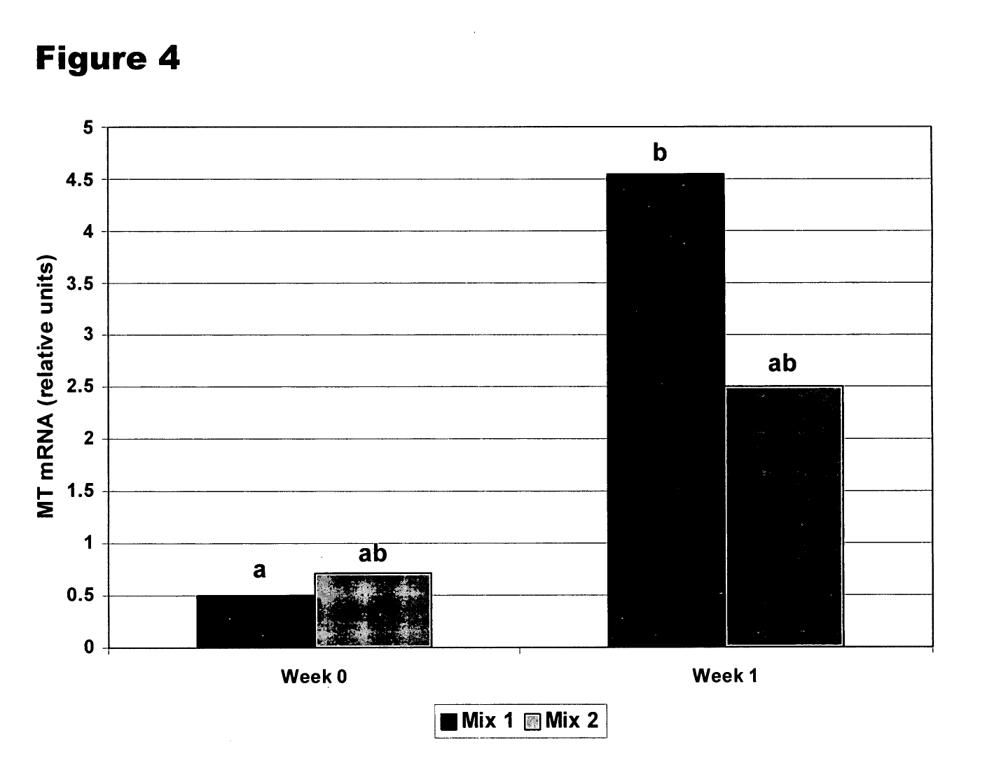

RNA was isolated and QRT-PCR was performed as described in Example 1, except bovine primers and probes, listed in Table 2, were used. MT expression was normalized to 18S rRNA expression, and the data are expressed relative to a cow liver reference sample. As shown in FIG. 4, liver MT expression was significantly higher in week 1 than week 0 in the animals fed Mix 1, but not in the animals fed Mix 2. These data indicate that the metal-HMTBA chelates were more bioavailable sources of metal than the specific amino acid complexes.

TABLE 2

Primers used for QRT-PCR in dairy cattle.

| | | |
|---|---|---|
| | | SEQ |
| | | ID |
| Primer Name | Sequence | NO |

| | | |
|---|---|---|
| bMT Forward Primer | 5′-CTGCTCCTGCCCCAC-3′ | 7 |
| bMT Reverse Primer | 5′-CAGCCCTGGGCACAC-3′ | 8 |
| bMT Taqman Probe | 5′- | 9 |
| | FAM-AGATGTCCCTCCTGCAAGAAGA- | |
| | BHQ1-3′ | |
| b18S Forward Primer | 5′-CACGGCCGGTACAGT-3′ | 10 |
| b18S Reverse Primer | 5′-CGCGAAGGGGGTCAG-3′ | 11 |
| b18S Taqman Probe | 5′-FAM- | 12 |
| | CTCGCTCCTCTCCTACTTGGATA- | |
| | BHQ1-3′ | |

What is claimed is:

1. A method for determining relative metal bioavailability of a metal source in at least one animal, the method comprising:
(a) administering the metal source to the animal;
(b) detecting the level of expression of a metal responsive biomarker present in a sample obtained from the animal and in a control sample; and
(c) comparing the level of expression of the metal responsive biomarker from the animal sample and the control sample, wherein a difference in the level of expression indicates the relative bioavailability of the metal source in the animal.

2. The method of claim 1, wherein the metal responsive biomarker mRNA is detected by quantitative real time polymerase chain reaction; the metal source comprises a metal selected from the group consisting of zinc, copper, iron, manganese, magnesium, calcium, potassium, selenium, cobalt, and chromium; and the metal responsive biomarker is selected from the group consisting of zinc transport proteins, metal-regulatory transcription factor, iron response element binding protein, ferritin, cytochrome c oxidase chaperone, ceruloplasmin, and superoxide dismutase.

3. The method of claim 2, wherein the control sample is a sample taken from the animal before the animal has been administered the metal source.

4. The method of claim 2, wherein the control sample is taken from an animal that has not been administered the metal source.

5. The method of claim 2, wherein the method is utilized to determine relative metal bioavailability of the metal source in a group of animals.

6. The method of claim 2, wherein the animal is selected from the group consisting of poultry, swine, sheep, cattle, goats, horses, freshwater animals, marine animals, game animals, and companion animals.

7. The method of claim 6, wherein the sample is from the animal's gastrointestinal tract and is collected at least 2 hours after the metal source has been administered to the animal.

8. The method of claim 7, wherein the metal source is a metal chelate or a metal salt comprising at least one hydroxy analog of methionine together with a metal ion selected from the group consisting of zinc ions, copper ions, manganese ions, magnesium ions, iron ions, calcium ions, potassium ions, chromium ions, cobalt ions, and selenium ions.

9. The method of claim 7, wherein the metal source is a complex of glycine and a metal ion selected from the group consisting of zinc ions, copper ions, manganese ions, magnesium ions, iron ions, calcium ions, potassium ions, chromium ions, cobalt ions, and selenium ions.

10. The method of claim 1, wherein the metal source comprises zinc or copper; the metal responsive biomarker is metallothionein mRNA that is detected by quantitative real time polymerase chain reaction; and a higher level of expression of metallothionein in the animal sample indicates the zinc source or the copper source is relatively bioavailable in the animal.

11. The method of claim 10, wherein the control sample is a sample taken from the animal before the animal has been administered the metal source.

12. The method of claim 10, wherein the control sample is taken from an animal that has not been administered the metal source.

13. The method of claim 10, wherein the method is utilized to determine relative zinc or copper bioavailability of the metal source in a group of animals.

14. The method of claim 10, wherein the animal is selected from the group consisting of poultry, swine, sheep, cattle, goats, horses, freshwater animals, marine animals, game animals, and companion animals; and the sample is from the animal's gastrointestinal tract and is collected at least 2 hours after the metal source has been administered to the animal.

15. The method of claim 14, wherein the metal source is a metal chelate comprising at least one hydroxy analog of methionine together with zinc ions or copper ions.

16. The method of claim 14, wherein the metal source is a complex of glycine and a metal ion selected from the group consisting of zinc ions and copper ions.

17. The method of claim 1, wherein the method is utilized to determine an optimal dosage of the metal source for at least one animal.

18. A method for comparing the relative metal bioavailability between a first metal source and a second metal source, the method comprising:
(a) administering the first metal source to a first animal and the second metal source to a second animal;
(b) detecting the level of expression of a metal responsive biomarker present in a first sample obtained from the first animal and in a second sample obtained from the second animal; and
(c) comparing the level of expression of the metal responsive biomarker from the first sample and the second sample, wherein a difference in the level of expression between the two samples indicates that the first metal source and the second metal source have different relative metal bioavailability.

19. The method of claim 18, wherein the method further comprises detecting the level of expression of a metal responsive biomarker in a control sample from a control animal that was not administered a metal source, and comparing the level of expression of the metal responsive biomarker from the first sample, second sample, and control sample.

20. The method of claim 19, wherein the metal responsive biomarker mRNA is detected by quantitative real time polymerase chain reaction; the first and second metal source comprise a metal selected from the group consisting of zinc, copper, manganese, magnesium, iron, calcium, potassium, selenium, cobalt, and chromium; and the metal responsive biomarker is selected from the group consisting of zinc transport proteins, metal-regulatory transcription factor, iron response element binding protein, ferritin, cytochrome c oxidase chaperone, ceruloplasmin, and superoxide dismutase.

21. The method of claim 19, wherein the method is utilized to compare relative metal bioavailability of the first metal source and the second metal source in at least two groups of animals.

22. The method of claim 19, wherein the relative bioavailability of more than two different metal sources is compared.

23. The method of claim 19, wherein the first metal source and the second metal source are administered at the same levels.

24. The method of claim 19, wherein the first metal source and the second metal source are administered at different levels.

25. The method of claim 19, wherein the first animal and the second animal are the same animal species selected from the group consisting of poultry, swine, sheep, cattle, goats, horses, freshwater animals, marine animals, game animals, and companion animals.

26. The method of claim 25, wherein the first sample and the second sample are from the animals' gastrointestinal tract and are collected at least 2 hours after the metal sources have been administered to the animals.

27. The method of claim 26, wherein at least one of the first metal source or the second metal source is a metal chelate or a metal salt comprising at least one hydroxy analog of methionine together with a metal ion selected from the group consisting of zinc ions, copper ions, manganese ions, magnesium ions, iron ions, calcium ions, potassium ions, chromium ions, cobalt ions, and selenium ions.

28. The method of claim 26, wherein at least one of the first metal source or the second metal source is a complex of glycine and a metal ion selected from the group consisting of zinc ions, copper ions, manganese ions, magnesium ions, iron ions, calcium ions, potassium ions, chromium ions, cobalt ions, and selenium ions.

29. The method of claim 18, wherein the first metal source and the second metal source comprise zinc or copper; the metal responsive biomarker is metallothionein mRNA that is detected by quantitative real time polymerase chain reaction; and a higher level of expression of metallothionein in one sample compared to the other sample indicates that the zinc source or copper source is relatively more bioavailable in one of the two metal sources.

30. The method of claim 29, wherein the method is utilized to compare relative metal bioavailability of the first metal source and the second metal source in at least two groups of animals.

31. The method of claim 29, wherein the relative bioavailability of more than two different metal sources is compared.

32. The method of claim 29, wherein the first animal and the second animal are the same animal species selected from the group consisting of poultry, swine, sheep, cattle, goats, horses, freshwater animals, marine animals, game animals, and companion animals.

33. The method of claim 29, wherein the first sample and the second sample are from the animals' gastrointestinal tract and are collected at least 2 hours after the metal sources have been administered to the animals.

34. The method of claim 33, wherein at least one of the first metal source or the second metal source is a metal chelate comprising at least one hydroxy analog of methionine together with zinc ions or copper ions.

35. The method of claim 33, wherein at least one of the first metal source or the second metal source is a complex of glycine and a metal ion selected from the group consisting of zinc ions and copper ions.

36. A method for determining the relative nutritional status of zinc or copper in an animal, the method comprising:
(a) detecting the level of metallothionein mRNA expression by quantitative real time polymerase chain reaction in a sample obtained from the animal and in a control sample; and
(b) comparing the level of metallothionein mRNA expression from the animal sample and the control sample, wherein a lower level of metallothionein mRNA in the animal sample versus the control sample indicates that the animal may have a zinc or copper deficiency.

37. The method of the 36, wherein the relative nutritional status of zinc or copper is determined for a group of animals.

38. The method of claim 36, wherein the animal is selected from the group consisting of poultry, swine, sheep, cattle, goats, horses, freshwater animals, marine animals, game animals, and companion animals.

39. The method of claim 38, wherein the sample is from the animal's gastrointestinal tract.


SEQUENCE LISTING (12 sequences)

SEQ ID NO: 1 - DNA, 15 bp, Artificial
  chicken
cctgtgctgggtcgt

SEQ ID NO: 2 - DNA, 15 bp, Artificial
  CHICKEN
tgctggccggttcct

SEQ ID NO: 3 - DNA, 16 bp, Artificial
  CHICKEN
tgctgctcctgctgcc

SEQ ID NO: 4 - DNA, 15 bp, Artificial
  CHICKEN
ggccgccggaatact

SEQ ID NO: 5 - DNA, 14 bp, Artificial
  CHICKEN
tcttgcgccggtcc

SEQ ID NO: 6 - DNA, 21 bp, Artificial
  CHICKEN
ccatgattaagagggacggcc

SEQ ID NO: 7 - DNA, 15 bp, Artificial
  CATTLE
ctgctcctgccccac

SEQ ID NO: 8 - DNA, 15 bp, Artificial
  CATTLE
cagccctgggcacac

SEQ ID NO: 9 - DNA, 22 bp, Artificial
  CATTLE
agatgtccctcctgcaagaaga

SEQ ID NO: 10 - DNA, 15 bp, Artificial
  CATTLE
cacggccggtacagt

SEQ ID NO: 11 - DNA, 15 bp, Artificial
  CATTLE
cgcgaagggggtcag

SEQ ID NO: 12 - DNA, 23 bp, Artificial
  CATTLE
ctcgctcctctcctacttggata